(19) United States
(12) Patent Application Publication
(10) Pub. No.: US 2004/0188637 A1
(43) Pub. Date: Sep. 30, 2004

(54) Infrared ray generating unit for sauna facilities

(76) Inventor: Seung Woo Lee, Mapo-ku (KR)

Correspondence Address: PARK & SUTTON LLP, 3255 WILSHIRE BLVD, SUITE 1110, LOS ANGELES, CA, 90010, US

(21) Appl. No.: 10/396,245

(22) Filed: Mar. 26, 2003

(51) Int. Cl. A61H 33/08; A61N 5/06

(52) U.S. Cl. 250/495.1; 250/504 R; 607/81; 607/91

(57) ABSTRACT

An infrared ray generating unit for sauna facility is provided. The unit includes a heater that generates a first infrared ray, a first reflector that surrounds the heater in a half circle, and a second reflector behind the first reflector. The first reflector has a reflecting layer and an infrared ray generating layer that is laminated to the reflecting layer. The reflecting layer reflects the first infrared ray. The reflecting layer is heated by heat transferred from the heater, and in turn, transfers heat to the infrared ray generating layer, and the infrared ray generating layer generates a second infrared ray with the heat transferred from the reflecting layer. The second reflector reflects the second infrared ray.

### BACKGROUND OF THE INVENTION

The present invention relates to an infrared ray generating unit for sauna facilities. More particularly, the invention relates to an infrared ray generating unit that is effective in generating infrared rays having wavelength useful for infrared ray sauna facilities.

The infrared ray is divided into the near infrared ray that has a wavelength from 0.76 to 1.5 micron, the middle infrared ray that has a wavelength from 1.5 to 5.6 micron, and the far infrared ray that has a wavelength from 5.6 to 1000 micron. Among these, the far infrared ray has a characteristic that may penetrate into human skin up to 40 mm, and resonates molecules that form the human cells; thereby the molecules generate heat by themselves.

An infrared ray sauna facility uses the far infrared ray as its heat source.

In contrast with a typical sauna device that heats air above 100 degree Celsius, the infrared ray sauna facility makes it possible to enjoy sauna at temperature as low as 40˜80 degree Celsius. Thus people who cannot enjoy the conventional high temperature sauna, including the old, the weak and children can enjoy sauna safely with the infrared sauna facility.

An infrared ray sauna facility includes a chamber into which a user can enter and sit. The chamber has a door to get into, and a seat for a user. A plurality of infrared ray generating units are installed on the inside wall of the chamber. A controller for controlling the infrared ray generating units is installed at the chamber. The user controls the temperature inside the chamber or the operating time of the facility with the controller.

The infrared ray generating unit is the core element of the infrared ray sauna facility. A high-efficiency sauna facility is possible to realize if a more efficient infrared ray generating unit is developed. Therefore, there have been efforts to improve the efficiency of the infrared ray generating unit, so that it radiates more infrared rays having desired wavelength with less energy.

### SUMMARY OF THE INVENTION

The present invention contrives to satisfy the need for an improved infrared ray sauna facility.

Therefore, an object of the invention is to provide an infrared ray generating unit that can radiate more infrared rays with less energy.

To achieve the above-described object, the invention provides an infrared ray generating unit that includes a heater that generates a first infrared ray, and a first reflector that is radially spaced from the heater by a predetermined distance and in an arc of at least 90 degrees. The first reflector has a reflecting layer and an infrared ray generating layer that is adjacent to the reflecting layer. The reflecting layer reflects the first infrared ray. The reflecting layer is heated by heat transferred from the heater, and transfers heat to the infrared ray generating layer, and the infrared ray generating layer generates a second infrared ray with the heat transferred from the reflecting layer.

Preferably, the reflecting layer and the infrared ray generating layer are laminated, and the bandwidth of the first infrared ray is different from the bandwidth of the second infrared ray.

The reflecting layer is made of stainless steel, and the infrared ray generating layer is made of a ceramic material.

The infrared ray generating unit may further include a second reflector that is radially spaced from the first reflector by a predetermined distance and in an arc of at least 90 degrees.

The infrared ray generating unit may further include a case that encloses the heater, the first reflector, and the second reflector, and the case has an opening for radiating the first infrared ray and the second infrared ray.

The heater includes a pipe and a heating wire enclosed in the pipe, and magnesium oxide is filled in the pipe.

A sauna facility having a chamber, in which a plurality of the infrared ray generating units are installed, is also provided.

The advantages of the present invention are that: (1) an infrared ray generating unit having higher efficiency is provided; and (2) the infrared ray generating unit has a simple construction.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view of an infrared ray generating unit for sauna facilities according to the present invention;

FIG. 2 is a cross-sectional view taken along the line 2-2 in FIG. 1;

FIG. 3 is a perspective view of an infrared ray sauna facility, in which the infrared ray generating units are installed; and

FIG. 4 is a schematic sectional view of the sauna facility.

### DETAILED DESCRIPTION OF THE PREFERRED EMBODIMENTS

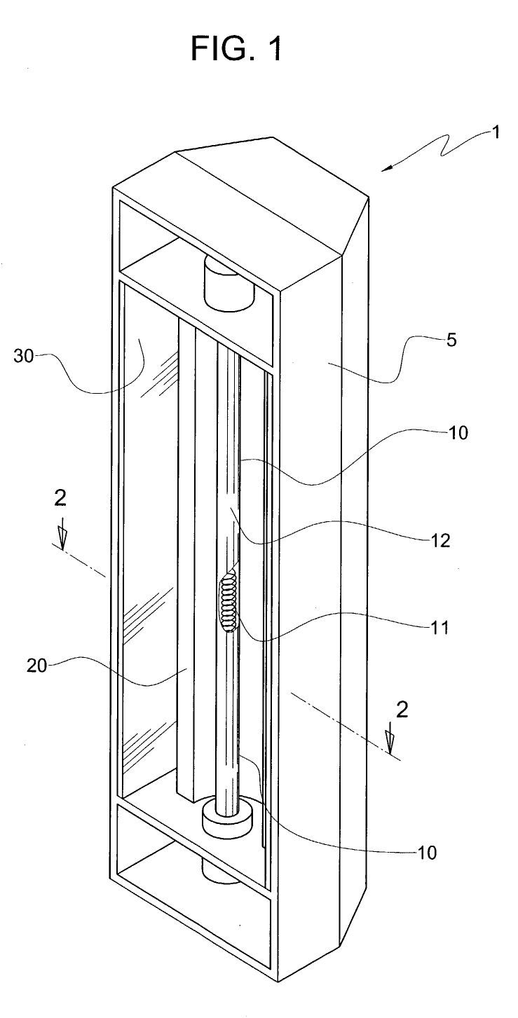

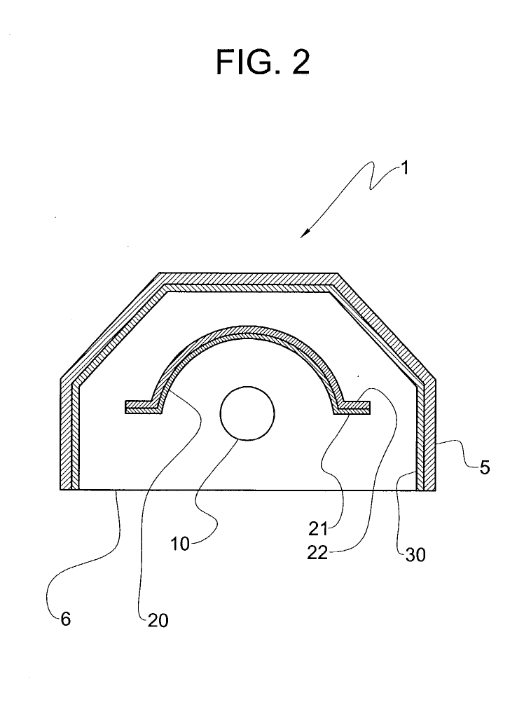

FIGS. 1 and 2 show an infrared ray generating unit 1 according to the present invention.

The infrared ray generating unit 1 includes a heater 10 that generates a first infrared ray and a first reflector 20 that is radially spaced from the heater 10 by a predetermined distance and in an arc of at least 90 degrees. In FIG. 2, the first reflector 20 is positioned behind the heater 10 and surrounds the heater 10 approximately in half circle.

The heater 10 includes a pipe 11, which was cut by a predetermined length, and a heating wire 12 enclosed in the pipe 11. The heating wire 12 is usually made of Ni-Cr alloy. Electric terminals are connected to the heating wire 12 at the ends of the pipe 11. Magnesium oxide is filled in the pipe 11 to fix the heating wire 12 and to improve heat transfer.

The first reflector 20 has a reflecting layer 21 and an infrared ray generating layer 22 that is laminated to the reflecting layer 21. The reflecting layer 21 faces the heater 10 and reflects the first infrared ray radiated from the heater 10 in the forward direction. The reflecting layer is heated by heat transferred from the heater 10, and in turn, transfers heat to the infrared ray generating layer 22. The infrared ray generating layer 22 generates a second infrared ray with the heat transferred from the reflecting layer 21.

The reflecting layer 21 is made of stainless steel, and the infrared ray generating layer 22 is made of a ceramic material that generates infrared ray having a predetermined wavelength.

A second reflector 30 is provided behind the first reflector 20. The second reflector 30 is radially spaced from the first reflector 20 by a predetermined distance and in an arc of at least 90 degrees. In FIG. 2, the first reflector 20 is circular, and the second reflector 30 has a shape of a partial polygon.

A case 5 is provided to enclose the heater 10, the first reflector 20, and the second reflector 30. The case has an opening 6 for radiating the first infrared ray and the second infrared ray.

When the heater 10 is activated by applying electricity on the heater, the heat generated by the heating wire 12 is transferred by radiation, conduction and convection to the pipe 11. The pipe 11 generates the first infrared ray having a predetermined wavelength. The first infrared ray is radiated forward toward a user sitting in a sauna facility, and also rearward from the heater 10. The first infrared ray radiated rearward is reflected by the reflecting layer 21 of the first reflector 20 and propagated toward the user. Heat is transferred to the first reflector 20 from the heater 10 by conduction and convection, etc. The transferred heat heats the infrared ray generating layer 22 to radiate the second infrared ray. The second infrared ray is reflected by the second reflector 30 and propagated forward to the user.

Preferably, the bandwidth of the first infrared ray, which is generated by the heater 10, is different from the bandwidth of the second infrared ray, which is generated by the infrared ray generating layer 22. The bandwidths may be selected by selection of materials for the pipe 11 and the infrared ray generating layer 22.

Infrared rays having different wavelengths penetrate into the skin up to different depths. Thus, for example, the first infrared ray may be used to penetrate deep into the skin and to vibrate molecules of human body so that the user should sweat. The second infrared ray may be used to penetrate into a different depth to vibrate other groups of molecules. More improved sauna effect is possible by differing wavelengths like this.

The second infrared ray is generated without applying electricity directly. Thus, with a given amount of energy, the amount of generated infrared rays is increased by the above construction. Thus, a high efficiency infrared ray generating unit is realized.

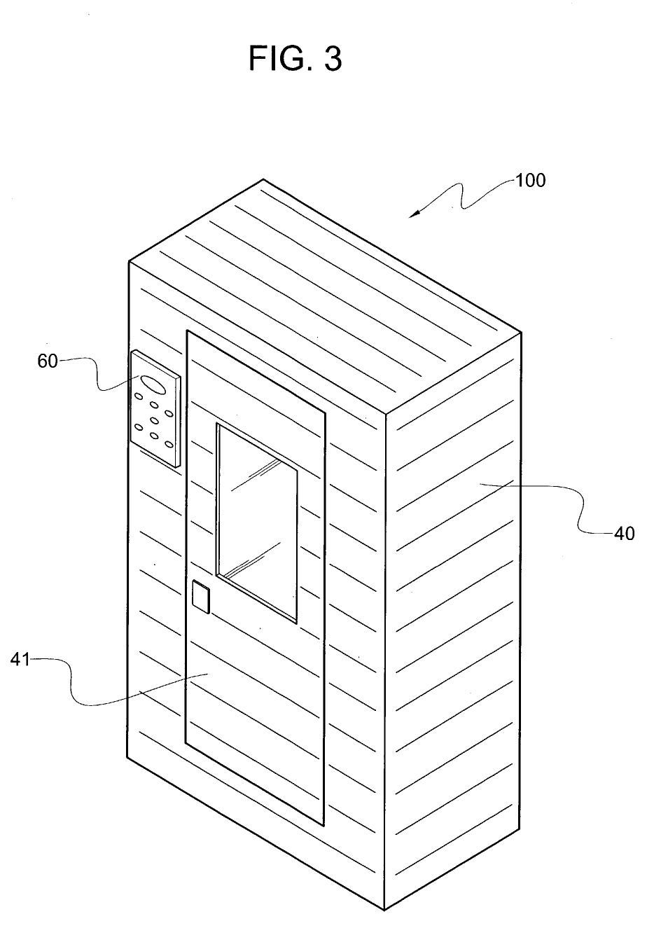

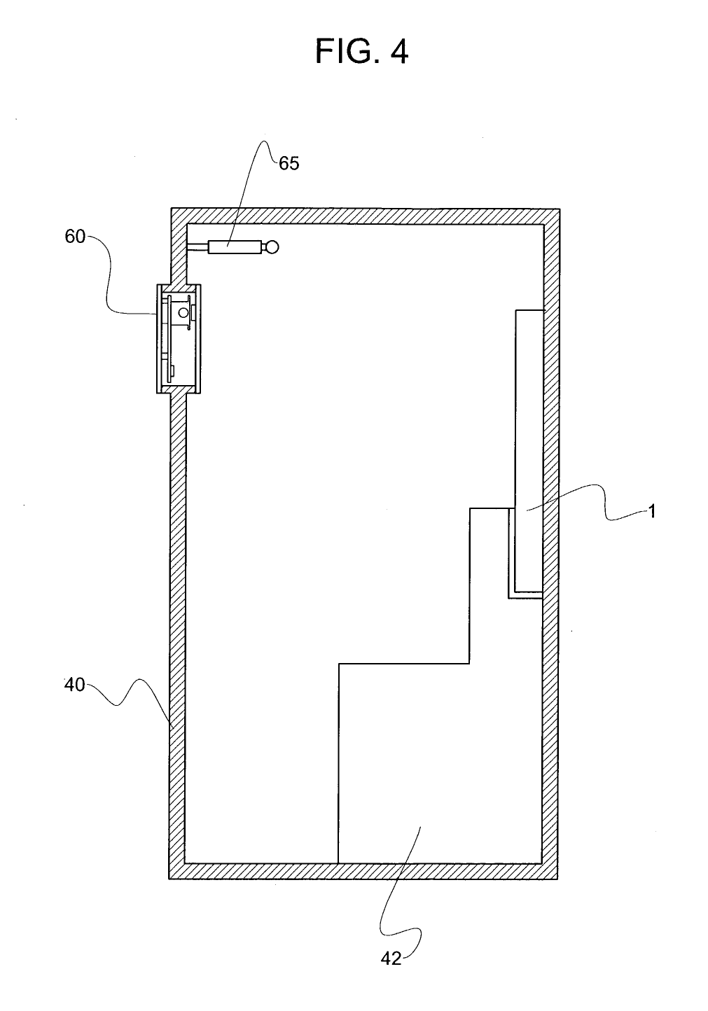

FIGS. 3 and 4 show an infrared sauna facility 100 having a chamber 40. A plurality of the infrared ray generating units 1 are installed in the chamber 40. The chamber 40 has a door 41, a control unit 60 for controlling the inside conditions of the chamber 40, a seat 42 for the user to sit on, and a temperature sensor 65.

The infrared sauna facility 100 can provide sauna effect at a temperature low as 50 degrees Celsius. Thus, the old, the weak and the children, who were not able to use a conventional high temperature sauna facility, can enjoy sauna safely.

Although the invention has been described in considerable detail, other versions are possible by converting the aforementioned construction. Therefore, the scope of the invention shall not be limited by the specification specified above.

What is claimed is:

1. An infrared ray generating unit comprising:
a) a heater that generates a first infrared ray; and
b) a first reflector that is radially spaced from the heater by a predetermined distance and in an arc of at least 90 degrees,
wherein the first reflector has a reflecting layer and an infrared ray generating layer that is adjacent to the reflecting layer,
wherein the reflecting layer reflects the first infrared ray,
wherein the reflecting layer is heated by heat transferred from the heater, and in turn, transfers heat to the infrared ray generating layer, and
wherein the infrared ray generating layer generates a second infrared ray with the heat transferred from the reflecting layer.

2. The infrared ray generating unit of claim 1, wherein the reflecting layer and the infrared ray generating layer are laminated.

3. The infrared ray generating unit of claim 1, wherein the bandwidth of the first infrared ray is different from the bandwidth of the second infrared ray.

4. The infrared ray generating unit of claim 1, wherein the reflecting layer is made of stainless steel.

5. The infrared ray generating unit of claim 1, wherein the infrared ray generating layer is made of a ceramic material.

6. The infrared ray generating unit of claim 1, further comprising a second reflector that is radially spaced from the first reflector by a predetermined distance and in an arc of at least 90 degrees.

7. The infrared ray generating unit of claim 6, wherein the reflecting layer and the infrared ray generating layer are laminated.

8. The infrared ray generating unit of claim 6, wherein the bandwidth of the first infrared ray is different from the bandwidth of the second infrared ray.

9. The infrared ray generating unit of claim 6, wherein the reflecting layer is made of stainless steel.

10. The infrared ray generating unit of claim 6, wherein the infrared ray generating layer is made of a ceramic material.

11. The infrared ray generating unit of claim 6, further comprising a case that encloses the heater, the first reflector, and the second reflector, wherein the case has an opening for radiating the first infrared ray and the second infrared ray.

12. The infrared ray generating unit of claim 11, wherein the heater comprises a pipe and a heating wire enclosed in the pipe.

13. The infrared ray generating unit of claim 12, wherein magnesium oxide is filled in the pipe.

14. A sauna facility having a chamber, the facility comprising a plurality of infrared ray generating unit installed in the chamber,
wherein each of the infrared ray generating units comprises a heater that generates a first infrared ray and a first reflector that is radially spaced from the heater by a predetermined distance and in an arc of at least 90 degrees,
wherein the first reflector has a reflecting layer and an infrared ray generating layer that is adjacent to the reflecting layer,
wherein the reflecting layer reflects the first infrared ray,
wherein the reflecting layer is heated by heat transferred from the heater, and in turn, transfers heat to the infrared ray generating layer, and
wherein the infrared ray generating layer generates a second infrared ray with the heat transferred from the reflecting layer.

15. The sauna facility of claim 14, wherein each of the infrared ray generating unit further comprises a second reflector that is radially spaced from the first reflector by a predetermined distance and in an arc of at least 90 degrees.

16. The sauna facility of claim 15, wherein the reflecting layer and the infrared ray generating layer are laminated.

17. The sauna facility of claim 15, wherein the bandwidth of the first infrared ray is different from the bandwidth of the second infrared ray.

18. The sauna facility of claim 15, wherein the reflecting layer is made of stainless steel.

19. The sauna facility of claim 15, wherein the infrared ray generating layer is made of a ceramic material.
